(19) United States
(12) Patent Application Publication
(10) Pub. No.: US 2003/0185856 A1
(43) Pub. Date: Oct. 2, 2003

(54) Method for the production of the egg containing anti-pathogenic bacteria specific antbodies(igy) and the yogurt and ice cream containing the igy

(76) Inventors: Nam-Hyung Lee, Seoul (KR); Jung-Soo Ryu, Daejeon (KR); Kwnag-Yong Jung, Daejeon (KR); Ban-Suk Baek, Gyeonggi-do (KR); Sun-Young Sunwoo, Seoul (KR)

Correspondence Address: Fleshner & Kim, PO Box 221200, Chantilly, VA, 20153-1200, US

(21) Appl. No.: 10/220,317

(22) PCT Filed: Mar. 31, 2001
(86) PCT No.: PCT/KR01/00550

(30) Foreign Application Priority Data
Jan. 5, 2001 (KR) 2001/000502
Feb. 23, 2001 (KR) 2001/0009367

(51) Int. Cl. A61K 39/116; A01K 31/19

(52) U.S. Cl. 424/203.1; 119/300

(57) ABSTRACT

The present invention provides the method for the production of the egg containing anti-pathogenic bacteria specific antibodies (IgY) preventing gastritis, diarrhea, and food poisoning by immunizing young hens with antigen proteins of E. coli causing enteritis, Helicobacter pylori causing gastritis, and Salmonella enteritidis and Salmonella typhimurium, causing food poisoning, simultaneously. This invention also relates to composition containing the specific IgY antibodies described above and the foodstuff such as the yogurt and ice cream containing the anti-pathogenic IgY.

Additionally, the present invention provides the separation method of the IgY containing protein powders from egg yolk. particularly, this separation method involves diluting egg yolk with water at 1:1 ratio and adding the appropriate amount of ammonium sulfate which enables water-soluble protein and phospholipid to separate.

### TECHNICAL FIELD

The present invention provides the method for the production of the egg containing anti-pathogenic bacteria specific antibodies (IgY) preventing gastritis, diarrhea, and food poisoning by immunizing young hens with antigen proteins of E. coli causing enteritis, Helicobacter pylori causing gastritis, and Salmonella enteritidis and Salmonella typhimurium, causing food poisoning, simultaneously, the composition containing the protein powders of the specific antibodies described above, mixed in the appropriate ratio, which produced by immunization with the four antigens separately, and the foodstuff processed with milk, such as the yogurt and ice cream, containing the anti-pathogenic bacteria specific antibodies (IgY).

Additionally, as the method for isolating the protein powders of the specific antibodies, the method for separating protein and phospholipid, particularly, proceeded in a process of diluting egg yolk with distilled water in 1:1 ratio, adding the appropriate amount of ammonium sulfate which enable water-soluble protein and phospholipid to separate, and the method for separating the pigment of egg-yolk and water-soluble protein, proceeded in a process of diluting those separated solution with distilled water, sitting in the certain temperature to precipitate and purify the proteins.

### BACKGROUND ART

According to reports about enterotoxigenic E. coli, a kind of an enteropathogenic E. coli which inhabits the intestinal tract of humans or animals causing diarrhea and abdominal pain, is known as enteritis pathogens not only for adult, but also for children. There are five kinds of diarrhea pathogens reported so far; Enteropathogenic E.coli (EPEC), Enteroinvasive E.coli (EIEC), Enterotoxigenic E.coli (ETEC), Enterohemorrhageic E.coli (EHEC), Enteroadhesive E.coli (EAEC). Adam isolated E.coli from the diarrhea patients of children and reproted those as pathogens responsible for enteritis in 1923. In the middle 1940's, diarrhea due to E.coli occurred in group usually in nursery in England. Since Neter named them Enteropathogenic E.coli as a first time. the group of E. coli has been called as pathogenic E.coli,

The prior art related to patent of E.1coli is summarized as following.

kim jungwoo et al(1999) filed the patent, of which object is providing the method for producing the egg yolk antibody from the immunoglobulin (IgY) in the egg yolk by utilizing ETEC K88 strain produced in the pig, and isolating them efficiently. Also, kim jungwoo et al(1999) filed the patent, of which object is providing the method for producing the egg yolk antibody from the immunoglobulin (IgY) in the egg yolk by utilizing ETEC K99 strain and K88 strain produced in the pig, and isolating them efficiently. Additionally, kim jungwoo et al(1999) filed the patent, of which object is providing the method for producing the egg yolk antibody from the immunoglobulin (IgY) in the egg yolk by utilizing ETEC 987p strain and K88 strain produced in the pig, and isolating them efficiently. Godama Yoshikashi(1998) filed the patent utilizing egg yolk antibody (IgY) obtained from hens immunized with the whole and vero toxoid of Enterohemorrhageic E.coli (EHEC) to prevent and treat the infection with Enterohemorrhageic E. coli (EHEC). Additionally, some of the patents has been summarized as following, which is related to the drug preventing infection with Helicobacter pylori causing duodenitis.

while the efforts to utilize the antigen-antibody reaction to treat gastritis and duodenitis has been continued, Coler et al. isolated immunoglobulin to treat gastritis and enteritis caused by Helicobacter pylori out of mammal milk secretion(1992). Taiyo gakakusha isolated specific antibody by antigenized and immunizing egg laying hens with antigenized Helicobacter pylori. The antigen utilized was Helicobacter pylori ATCC strain 43504, 43506, 43579, and 43629. Recently among infectious disease common for animal and mammals such as S. enteritidis, S. typhimurium, and enteropathogenic E. coli were raising a great social trouble in increasing rate. Among causing of the highest occurring frequency is the food poisoning, bacteria-attributable food poisoning, The main pathogens causing food poisoning are Salmonella, Vibrio cholerae or staphylococcus, Among enteric bacterium causing food poisoning in humans, Salmonella shows highest occurrences. Since the food poising patient caused by Salmonella is 37.7% of total. Salmonella is the major cause of food poisoning, While food poisoning caused by Vibrio cholerae occurred during summer, those caused by Salmonella are reported throughout year long, which shows the importance of control. The suffers from Salmonella-attributable food poisoning continuously occurs in South America and Europe besides North America. Moreover the ratio of the occurrence and maintaining the pathogens are increasing due to the augment of mass-transportation of beef, environmental pollution, mass dining such as in school cafeteria. The ultimate removal of Salmonella is impossible, since 2400 serotypes of Salmonella except Salmonella pullorum, Salmonella gallmarum infect more than one animals at any condition without certain limits for host. Salmonella enteritidis and Salmonella typhimurium has been reported as major causes of food poisoning (KFDA, the journal of epidemic occurrence information, Kim, Hohoon(1997)). Usually, the major course of infection is on egg infection, but in egg infection can be the course of infection for Salmonella. Especially, for the case of Salmonella enteritidis and Salmonella typhimurium, it was reported that the infection in the ovary transmitted to the egg yolk the in egg infection, which shows the importance of in egg infection. Therefore, the prevention of in egg infection can be the key to the prevention of Salmonella-attributable food poisoning by blocking the epidemic process.

As a prior art related to Salmonella, Song Kyungbin (2000) filed the patent about the expression of fusion protein of ATFA subunit of Salmonella enteritidis with maltose binding protein in E. coli. The object of this invention is to be used for making the diagnosis kit and developing vaccine for Salmonella infection which is major disease for chickens. As mentioned above, a variety of methods such as utilizing antibiotics and natural resources have been tried to suppress the growth of Helicobacter and Salmonella without having the certain conclusion of drug efficacy evaluation. As the problem of food poisoning is raised every year, producing egg containing antibody against Salmonella and other antibodies can be suggested as more effective and radical prevention method. Generally, producing antibody against certain pathogens and proceeding the treatment by utilizing antibody has been known for the most ideal way for the treatment. Producing and isolating the specific antibodies for each pathogen are remained as to be solved in industrial scale,

As the prior art related to other invention, there were some inventions related to the separation method of the protein and phospholipid out of egg yolk containing the one of the specific immunoprotein, IgY. The proteins of egg yolk constituted 15˜17% of the whole, comprise the α,β,γ-livetin as major kinds. The IgG class of γ-livetin is a specific immunoprotein present in egg yolk(Poson et al., 1980), known as IgY in general. IgY is the specific immunoprotein which can be taken orally (McBee et al., 1979). For the separation of IgY, the first step is removing the lipid and lipoprotein in the egg yolk.

As methods to remove those, the separation method of lipoprotein by ultra centrifugation (Bade, 1984), the removal method of lipid utilizing organic solvent (Poison et al., 1980), the precipitation method of lipoprotein utilizing polyethyleneglycol(PEG) and sodium dextran sulfate(SDS)(Hachida, 1993). Especially, the method utilizing the natural polysaccharide “carrageenan”, which has been used widely as thickening stabilizer for a foodstuff and strongly activates the coagulation of egg yolk lipoprotein (Hansen et. al, 1998).

The prior arts described above has the limitation of low efficiency and high cost on a industrial scale for the purification and separation in mass production, even though they have been utilized to separate the water-soluble protein and lipid from egg yolk.

### DISCLOSURE OF INVENTION

With the aim of solving the problems of prior arts described above, it is an object of the present inventions to provide the method for the production of the egg containing anti-pathogenic bacteria specific antibodies (IgY) containing anti-E.coli IgY and anti-Helicobacter pylori IgY and anti-Salmonella enteritidis IgY and anti-Salmonella typhimurium IgY simultaneously, and the foodstuff processed with milk, such as the yogurt and ice cream, containing the mixtures of anti-pathogenic bacteria specific antibodies (IgY).

Additionally, as a separation method of water-soluble protein from egg yolk, this invention allows easy separation and purification in a large scale by using nontoxic ammonium sulfate and distilled water without ultracentrifugation and precipitant, which is different from the prior arts.

To achieve the technical subject described above, the present invention relates to;

(1) the method for the production of the egg containing anti-pathogenic bacteria specific antibodies (IgY) containing anti-E.coli IgY and anti-Helicobacter pylori IgY and anti-Salmonella enteritidis IgY and anti-Salmonella typhimurium IgY simultaneously, produced by immunizing chicks by injecting with 1 ml of mixed antigen proteins of E. coli, Helicobacter pylori, Salmonella enteritidis and Salmonella typhimurium, emulsified with aluminum hydroxide in a certain ratio at a first time, and 1 ml each of emulsified mixed antigen proteins using the emulsifying adjuvant ISA25 for two times by 1 week interval, again immunizing the grown egg laying hens with 0.5 ml each of emulsified mixed antigen proteins using the emulsifying adjuvant ISA25 in the same ratio for two times by 3 month interval as a total of 5 times.

In detail, the method for the production of the egg containing anti-mixed pathogenic bacteria specific antibodies (IgY) comprises the steps of administering chicks with 1 ml of the mixed antigen proteins of E. coli, Helicobacter pylori, Salmonella enteritidis and Salmonella typhimurium, emulsified with aluminum hydroxide in a certain ratio at a first time, which comprise 0.15 ml antigen of nonliving E. coli 4.0×10⁸/ml, 0.1 ml antigen of nonliving Salmonella enteritidis 4.0×10⁸/ml, 0.10 ml antigen of nonliving Salmonella typhimurium 4.0×10⁸/ml, and 0.35 ml antigen of nonliving Helicobacter pylori 4.0×10⁸/ml, emulsified with 0.3 ml of aluminum hydroxide. in the ratio of E. coli:Salmonella enteritidis:Salmonella typhimurium:Helicobacter pylori:emulsifying adjuvant=1.5:1:1:3.5:3 and for second and third injection, 1 ml of mixed antigen proteins diluted with the emulsifying adjuvant ISA25 in 1:1 ratio for two times with 1 week interval, again immunizing the grown egg laying hens with 0.5 ml each of emulsified mixed antigen proteins comprising 0.15 ml antigen of nonliving E. coli 2.0×10⁸/ml, 0.10 ml antigen of nonliving Salmonella enteritidis 2.0×10⁸/ml, 0.10 ml antigen of nonliving Salmonella typhimurium 2.0×10⁸/ml, and 0.35 ml antigen of nonliving Helicobacter pylori 2.0×10⁸/ml, emulsified with 0.3 ml of aluminum hydroxide in the same ratio for two times by 3 month interval as a total of 5 times.

(2) another method for the production of the egg containing anti-mixed pathogenic bacteria specific antibodies (IgY) produced by immunizing chicks by administering with 1 ml of mixed antigen proteins of E. coli, Helicobacter pylori, Salmonella enteritidis and Salmonella typhimurium, emulsified with Adjuvant complete Freund's in a certain ratio at a first time, and 1 ml each of emulsified mixed antigen proteins using Adjuvant incomplete Freund's for two times by 2 week interval, again immunizing the grown egg laying hens with 0.5 ml each of the emulsified mixed antigen proteins in the same ratio for two times by 3 month interval as a total of 5 times.

(3) Another method for the production of the egg containing anti-pathogenic bacteria specific antibodies (IgY) differed by immunizing with E. coli, Salmonella enteritidis and Salmonella typhimurium only, instead of 4 pathogens, whereas it utilized same egg production methods as (1), (2).

(4) Another invention related to the isolation methods of specific immunoproteins comprising the steps of collecting the 35 g egg yolk separated from the egg containing mixed anti-pathogenic bacteria specific antibodies (IgY) simultaneously, to the 250 ml bottle, mixing with 35 ml of alkali ionic water(pH9), sitting for 24 hours on 5-10° C., adding 18 vol of alkali ionic water(pH10) (1260 ml), sitting for 48 hours, separating the supernatant containing water-soluble specific immunoprotein, concentrating the supernatant using ultra filtration system and freeze-drying the concentrated supernatant.

Another isolation methods of specific immunoproteins comprising the steps of diluting the egg yolk separated from the egg containing mixed anti-pathogenic bacteria specific antibodies (IgY) with distilled water in a certain ratio, adding ammonium sulfate to the diluents of egg yolk and distilled water to separate the water-soluble specific immunoprotein and phospholipid, sitting for certain time, diluting the supernatant collected after removing the upper lipid layer again, sitting for certain times, and isolating/purifying specific immunoprotein, more preferentially, wherein the diluting ratio of egg yolk and distilled water is 1:1, or wherein the amount of ammonium sulfate added is 3˜10%, 5%˜6% preferred.

(5) Another invention related to the mixed composition of anti-pathogen specific immunoprotein powder produced by mixing the water-soluble specific immunoproteins each (crude IgY)(E. coli:Salmonella enteritidis:Salmonella typhimurium:Helicobacter pylori) isolated from the eggs containing specific immunoproteins separately. The eggs containing specific immunoproteins separately are produced by the method immunizing different chicks by administering with 1 ml (10⁸/ml) antigen proteins each of E. coli, Helicobacter pylori, Salmonella enteritidis and Salmonella typhimurium, emulsified with emulsifying adjuvant in a 1:1 ratio separately at a first time, and 1 ml each of the antigen proteins emulsified with the emulsifying adjuvant Adjuvant complete Freund's for two times by 2 weeks interval, again, which comprise the 0.5 ml antigen of nonliving E. coli 2.0×10⁸/ml and the 0.5 ml, of Adjuvant complete Freund's, the antigen proteins 0.5 ml antigen of nonliving Salmonella enteritidis 2.0×10⁸/ml and the 0.5 ml of Adjuvant complete Freund's, 0.5 ml antigen of nonliving Salmonella typhimurium 2.0×10⁸/ml and the 0.5 ml of Adjuvant complete Freund's, and 0.5 ml antigen of nonliving Helicobacter pylori 2.0×10⁸/ml and the 0.5 ml of Adjuvant complete Freund's in the 1:1 ratio accordingly, immunizing the grown egg laying hens with 0.5 ml each of emulsified antigen proteins, which comprise the 0.5 ml antigen of nonliving E. coli 2.0×10⁸/ml and the 0.5 ml of Adjuvant incomplete Freund's, the antigen proteins 0.5 ml antigen of nonliving Salmonella enteritidis 2.0×10⁸/ml and the 0.5 ml of Adjuvant incomplete Freund's, 0.5 ml antigen of nonliving Salmonella typhimurium 2.0×10⁸/ml and the 0.5 ml of Adjuvant incomplete Freund's, and 0.5 ml antigen of nonliving Helicobacter pylori 2.0×10⁸/ml in and the 0.5 ml of Adjuvant incomplete Freund's in the 1:1 ratio accordingly for two times by 3 month interval as a total of 5 times.

Another invention related to the mixed composition of anti-pathogen specific immunoprotein powder produced by mixing the water-soluble specific immunoproteins each (crude IgY)(E. coli:Salmonella enteritidis:Salmonella typhimurium:Helicobacter pylori) isolated from the eggs containing specific immunoproteins separately. The eggs containing specific immunoproteins separately are produced by the method immunizing different chicks by administering with 1 ml (10⁸/ml) antigen proteins each of E. coli, Helicobacter pylori, Salmonella enteritidis and Salmonella typhimurium, emulsified with emulsifying adjuvant in a 1:1 ratio separately at a first time, and 1 ml each of the antigen proteins emulsified with the emulsifying adjuvant ISA25 for two times by 2 weeks interval, again, which comprise the 0.5 ml antigen of nonliving E. coli 2.0×10⁸/ml and the 0.5 ml of emulsifying adjuvant ISA25, the antigen proteins 0.5 ml antigen of nonliving Salmonella enteritidis 2.0×10⁸/ml and the 0.5 ml of emulsifying adjuvant ISA25, 0.5 ml antigen of nonliving Salmonella typhimurium 2.0×10⁸/ml and the 0.5 ml of emulsifying adjuvant ISA25, and 0.5 ml antigen of nonliving Helicobacter pylori 2.0×10⁸/ml and the 0.5 ml of emulsifying adjuvant ISA25 in the 1:1 ratio accordingly, immunizing the grown egg laying hens with 0.5 ml each of emulsified antigen proteins, which comprise the 0.5 ml antigen of nonliving E. coli 2.0×10⁸/ml and the 0.5 ml of emulsifying adjuvant ISA25, the antigen proteins 0.5 ml antigen of nonliving Salmonella enteritidis 2.0×10⁸/ml and the 0.5 ml of emulsifying adjuvant ISA25, 0.5 ml antigen of nonliving Salmonella typhimurium 2.0×10⁸/ml and the 0.5 ml of emulsifying adjuvant ISA25, and 0.5 ml antigen of nonliving Helicobacter pylori 2.0×10⁸/ml and the 0.5 ml of emulsifying adjuvant ISA25 in the 1:1 ratio accordingly for two times by 3 month interval as a total of 5 times.

(6) Another method related to the production of the foodstuff processed with milk, such as the yogurt and ice cream and food additives containing the anti-pathogenic bacteria specific antibodies (IgY) simultaneously, produced by the methods described in (1), (2). (3), (4). and containing the mixed composition of anti-pathogen specific immunoprotein powder produced as described in (5).

The advantage of this invention according to the methods described above is:

the foodstuff processed with milk, such as the yogurt, ice cream and food additives containing the anti-pathogenic bacteria specific antibodies (IgY) is effective for the prevention of gastritis and enteritis by the ingestion. Also, by using the eggs containing anti-Salmonella IgY, the sterilization can be minimized and the secondary infection by Salmonella can be prevented. Even the contamination occurred in part, the anti-Salmonella IgY will mollify the problem of food toxication.

For the effect of another invention about the isolation method of specific immunoproteins, the loss of the potency of IgY can be minimized and only little amount of the ammonium sulfate can be utilized for the mass production, which are more advantageous than the prior art utilizing the organic solvent and precipitant to separate the lipid and water-soluble protein. This invention made it possible to obtain the 97% productivity after diluting with distilled water and sitting overnight in 4° C., to remove the color of egg-yolk completely, and to separate in complete without utilizing ultracentrifuge, which attribute to cost down.

The present inventions is hereinafter described in more detail by means of the following working examples, figures, and tables, but the inventions is not limited by these examples.

### BRIEF DESCRIPTION OF THE DRAWINGS

FIG. 1a is the change of the potency of anti-Helicobacter pylori specific IgY after immunizing with the mixture containing E. coli, Helicobacter pylori, Salmonella enteritidis, and Salmonella typhimurium.

FIG. 1b is the change of the potency of anti-Salmonella specific IgY after immunizing with the mixture containing E. coli, Helicobacter pylori, Salmonella enteritidis, and Salmonella typhimurium.

FIG. 1c is the average potency of anti-Helicobacter pylori specific IgY after immunizing with the mixture containing E. coli, Helicobacter pylori, Salmonella enteritidis, and Salmonella typhimurium.

FIG. 1d is the average potency of anti-Salmonella specific IgY after immunizing with the mixture containing E. coli, Helicobacter pylori, Salmonella enteritidis, and Salmonella typhimurium.

FIG. 1e is the change of the potency of anti-Helicobacter pylori specific IgY after immunizing with the mixture containing E. coli, Salmonella enteritidis, and Salmonella typhimurium.

FIG. 1f is the change of the potency of anti-E. coli specific IgY after immunizing with the mixture containing E. coli, Salmonella enteritidis, and Salmonella typhimurium.

FIG. 1g is the average potency of anti-Salmonella specific IgY after immunizing with the mixture containing E. coli, Salmonella enteritidis, and Salmonella typhimurium.

FIG. 1h is the average potency of anti-E. coli specific IgY after immunizing with the mixture containing E. coli, Salmonella enteritidis, and Salmonella typhimurium.

FIG. 2a is the drawing about the method for the separation and purification of water-soluble specific immunoprotein from the egg yolk.

FIG. 2b shows the separation of water-soluble specific immunoprotein and lipoprotein of the egg yolk by ammonium sulfate treatment.

FIG. 2c is the change of the potency of water-soluble specific immunoprotein IgY by ammonium sulfate treatment.

FIG. 2d is the change of the potency of water-soluble specific immunoprotein IgY according to dilution factor.

FIG. 2e is the change of the potency of water-soluble specific immunoprotein IgY according to dilution factor after homogenization.

FIG. 2f is the change of the potency of water-soluble specific immunoprotein IgY by concentration and dialysis

FIG. 2g is the change of the potency of water-soluble specific immunoprotein IgY after production of mayonnaise.

FIG. 2h is about the purity of water-soluble specific immunoprotein IgY utilizing PAGE after purification.

### BEST MODE FOR CARRYING OUT THE INVENTION

### Example 1

1. Isolation of Enteropathogenic E. coli and Production of Antigen

a. Isolation and Identification of Enterotoxigenic E. coli (ETEC) from Human

The pathogenic E. coli used in this invention is isolated from human. The enterotoxigenic E. coli (ETEC) utilized as antigen is isolated from the diarrhea of children. The isolation of the E. coli is proceeded as following. The diarrhea of children was streaked on the serum agar plate. The colony of enterotoxigenic E. coli (ETEC) showing α-hemolytic was selected after incubation for 18 hours on 37° C. The selected colonies were grown into pinky colonies in the MacConkey agar, and were grown into metallic green colonies in the EMB agar, which is characteristics of E. coli.

b. Examination of the Enter Toxin Producing Ability

The enterotoxin producing ability of E. coli. were examined by polymerase chain reaction (PCR). The primers specific for the STa1 gene of heat stable toxin(ST) and the LTh gene for heat lable toxin(LT) were utilized for the multiplex PCR amplification.

Primer for ST toxin were

sense primer; CCCCTCTTTTAGTCAGTC

anti-sense primer; CCAGCACAGGCAGGATTACA

These were designed to produce 165 bps product.

Primer for LT toxin were

sense primer; CAGACTATCAGTCAGAGGTTG

anti-sense primer; TTCATACTGATTGCCGCA

These were designed to produce 417 bps product.

The condition of PCR were as following; Pre-denaturation 95° C. 5 minute, Denaturation 94° C. 1 minute, Annealing 56° C. 1 minute, Elongation 72° C. 1 minute, Post-elongation 72° C. 10 minute. The PCR product were examined by electrophoresis on the 2% agarose gel. The isolated E. coli. strain used in this invention was checked to producing toxin. This E. coli. strain were named as EB-E01.

c. Production of the Enterotoxigenic E. coli (ETEC) Antigen.

The enterotoxigenic E. coli (ETEC) strain was inoculated on the Trypticase soy agar, incubated for 18 hours on the 37° C. incubator. One colony were selected, inoculated on the 5 ml of Trypticase soy broth and grown for 2 hours. The culture was inoculated to the large amount of Trypticase soy broth and incubated for 48 hours in the 37° C. without shaking. Formaldehyde were added into the culture to become 6% of total volume and immortalized for 24 hours at room temperature. The immortalized culture were centrifuged for 20 minute at 4000 rpm. The collected precipitant were washed three times with phosphate buffered saline(PBS)(pH7.2). The collected culture were suspended in the PBS up to O.D.(Oculus Dexter) 1.2˜1.3 at 410 nm to be used.

2. Production of the Helicobacter pylori Antigen.

a) Helicobacter pylori

Helicobacter pylori was taken from American Type Culture Collection(ATCC43504). The test culture were grown on the Trypticase soy agar; BBL with 10% sheep serum, passed every 3 to 5 week and incubated for 10% CO₂ incubator at 37° C. The test culture was examined by microscopically method and the urease activity test.

b) Examination of Urease Activity

The examination of Urease activity were done by using urease test broth containing urea and phenol red. The urease activity were determined by measuring the O.D. at 410 nm of the mixture of urea broth, culture media, and test treatment at 540 nm.

c) Morphological Test of Bacterium

The morphological change of the bacterium cultured for 3-5 days with bare eye, the colonies were streaked on the slide glass, stained by gram staining and examined under the microscope(×1,000) to determine whether the morphology is maintained as the curved form or changed into coccoid form.

d) Production of the Helicobacter pylori Antigen.

The cultured Helicobacter pylori were collected by cell collector, suspended on the Saline, and immortalized by heating for 30 minute at 60° C. water bath. The culture were collected by centrifugation 10,000×g for 15 minute, and the steps of suspension and the centrifugation were repeated 3 times to remove the media. The number of bacterium immortalized were counted by hematocytometer.

3. Production of the Salmonella enteritidis Antigen, and Salmonella typhimurium. Antigen.

The Salmonella enteritidis was taken from the Korean Culture Centre of Micro-organisms No. 12021(KCCM 12021), and the Salmonella typhimurium was obtained from the Korean Culture Centre of Micro-organisms No.11863(KCCM 11863).

The Salmonella enteritidis and Salmonella typhimurium, strain was inoculated on the Trypticase soy agar, incubated for 18 hours on the 37° C. incubator. One colony were selected, inoculated onto the large amount of Trypticase soy broth and incubated for 48 hours in the 37° C. without shaking. Formaldehyde were added into the culture to become 0.2% of total volume and immortalized for 24 hours at room temperature. The immortalized culture were centrifuged for 20 minute at 4000 rpm. The collected precipitant were washed three times with saline (pH7.2). The collected culture were stored at −70° C. to be used.

4. Immunization by Injection of the Mixed 4 Antigens into Chicks and Boosting Injection into Egg Laying Hens.

a) The number of the bacterium injected into the 12 weeks old chicks was 10⁸/ml. The ratio was E. coli:Salmonella enteritidis:Salmonella typhimurium:Helicobacter pylori:emulsifying adjuvant=1.5:1:1:3.5:3. In detail, The first injection were done with the mixed antigen proteins comprising 0.15 ml antigen of nonliving E. coli 4.0×10⁸/ml, 0.10 ml antigen of nonliving Samonella enteritidis 4.0×10⁸/ml, 0.10 ml antigen of nonliving Salmonella typhimurium 4.0×10⁸/ml, and 0.35 ml antigen of nonliving Helicobacter pylori 4.0×10⁸/ml, emulsified with 0.3 ml of aluminum hydroxide. From the second time as boosting injections, the mixed antigen emulsified with emulsifying adjuvant ISA25 were used. Two times of injection with 1 ml were done into chicks with two week interval. 28 weeks egg laying hens were injected with 0.5 ml each of emulsified mixed antigen proteins for two times by 3 month interval as a total of 5 times.

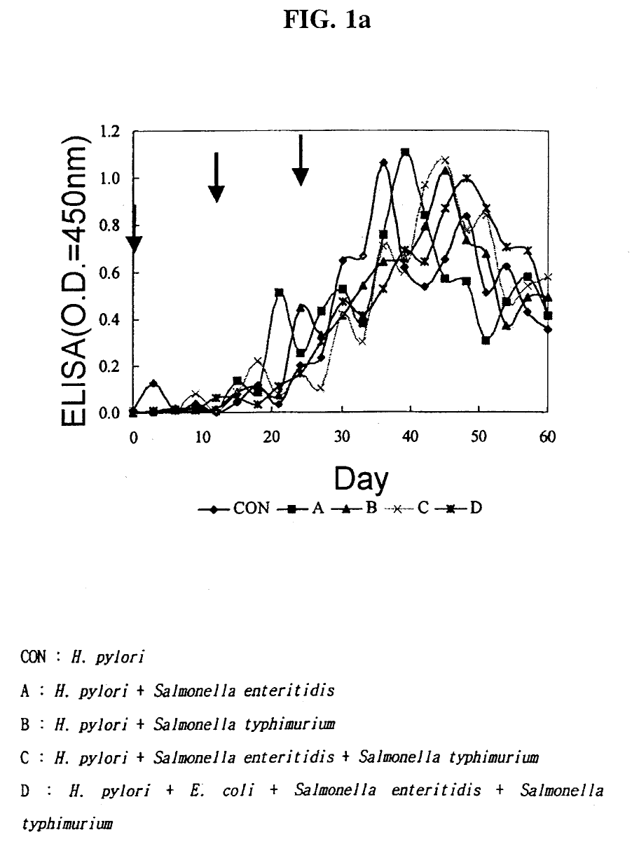

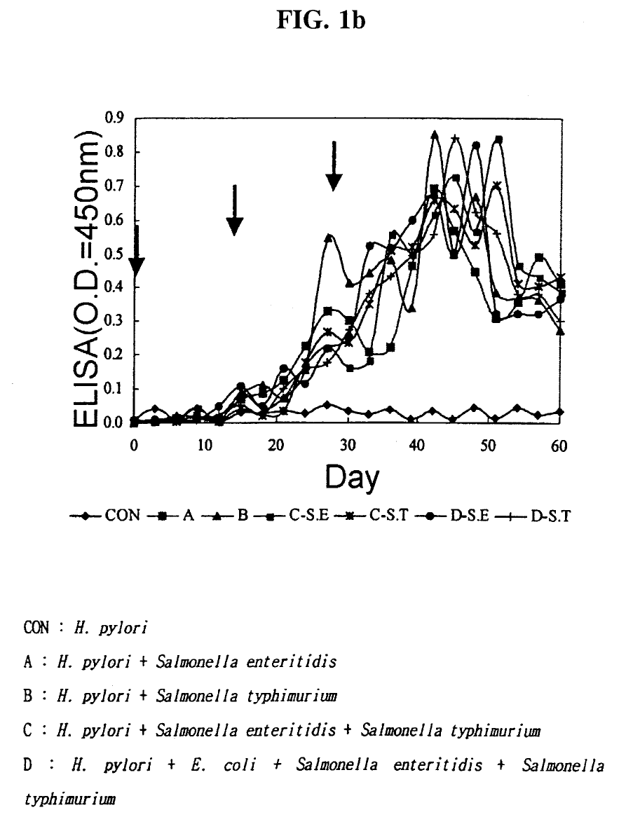

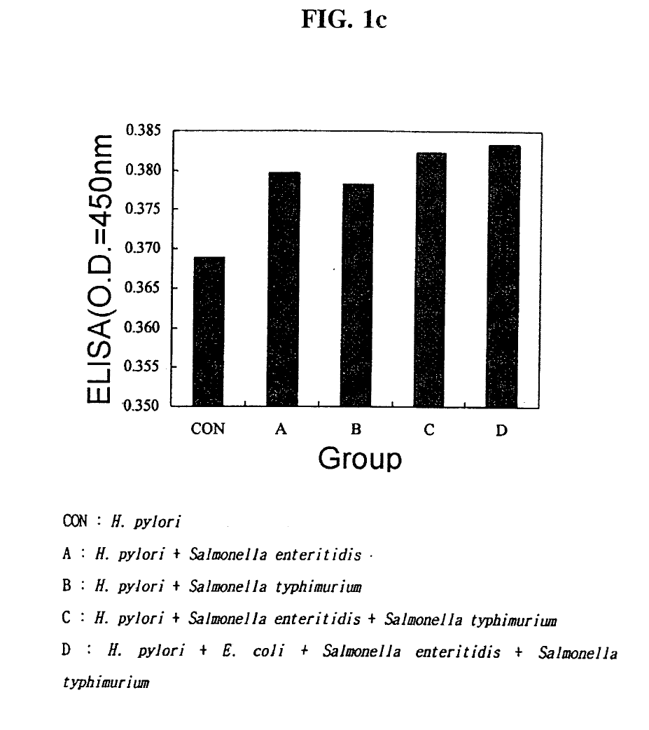

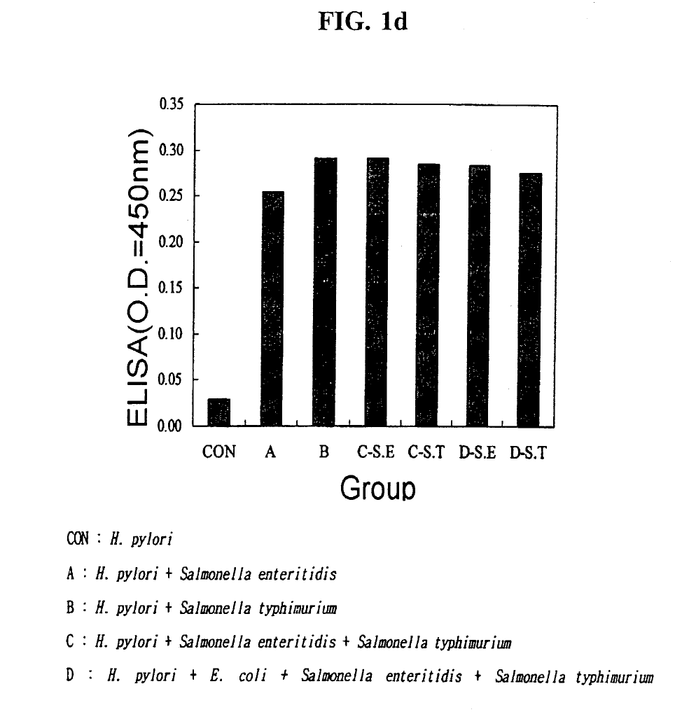

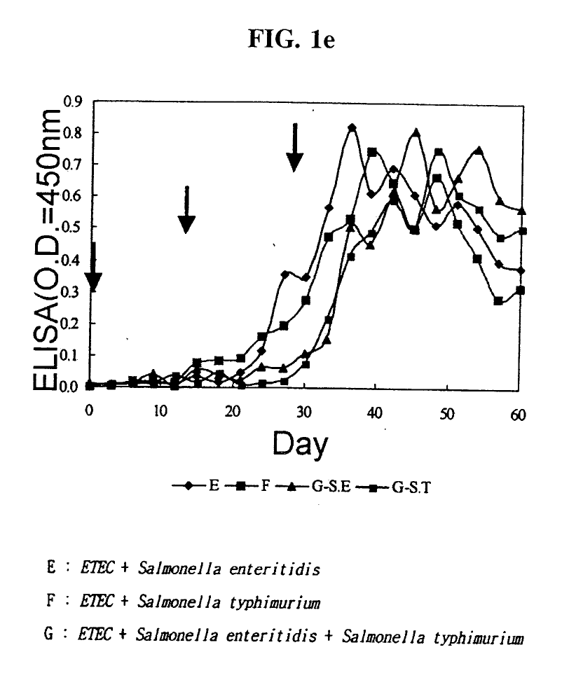

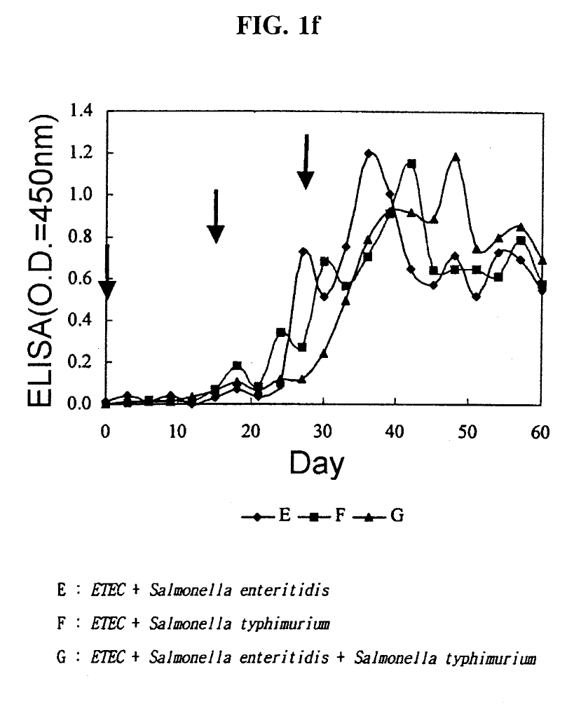

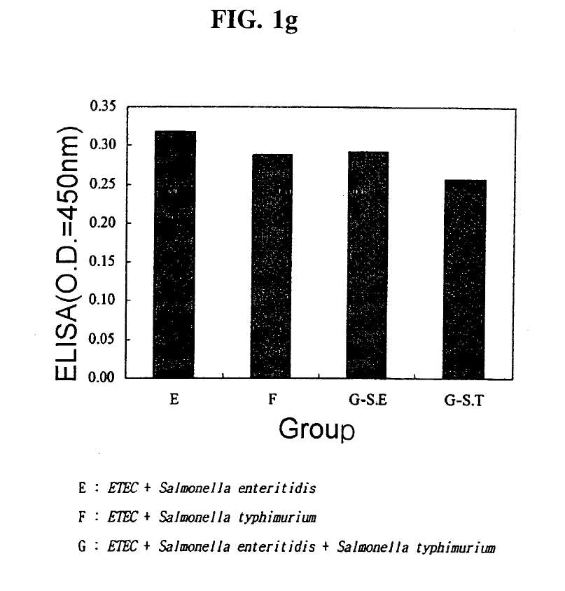

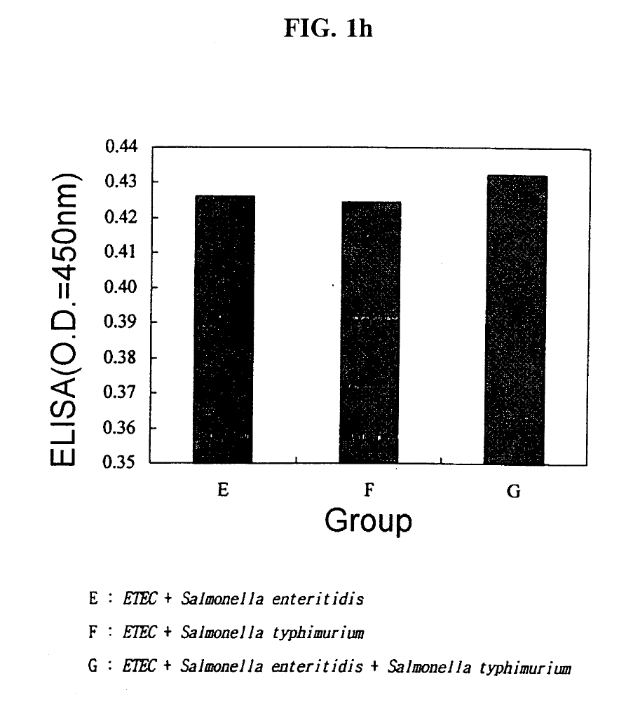

b) When emulsifying adjuvant ISA25 were used., the average potency of anti-Salmonella specific IgY and anti-Helicobacter pylori specific IgY of the group immunized with the antigen mixture containing more than two antigens were significantly higher than control group immunized with one of antigens (FIGS. 1a,1b,1c,1d).

c) The number of the bacterium injected into the 12 week old chicks was 10⁸/ml. The ratio was E. coli:Salmonella enteritidis:Salmonella typhimurium:Helicobacter pylori:emulsifying adjuvant=1.5:1.1:3.5:3. In detail, the first injection were done with the mixed antigen proteins comprising 0.15 ml antigen of nonliving E. coli 4.0×10⁸/ml, 0.10 ml antigen of nonliving Salmonella enteritidis 4.0×10⁸/ml, 0.10 ml antigen of nonliving Salmonella typhimurium 4.0×10⁸/ml, and 0.35 ml antigen of nonliving Helicobacter pylori 4.0×10⁸/ml, emulsified with 0.3 ml of Adjuvant complete Freund's. From the second time as boosting injections, the mixed antigen emulsified with Adjuvant incomplete Freund's were used. Two times of injection with 1 ml were done into chicks with two week interval. 28 weeks egg laying hens were injected with 0.5 ml each of emulsified mixed antigen proteins for two times by 3 month interval as a total of 5 times. As a result, the eggs containing anti-E.coli IgY and anti-Helicobacter pylori IgY and anti-Salmonella enteritidis IgY and anti-Salmonella typhimurium IgY, simultaneously, were produced from the hens immunized with the mixture of antigens emulsified.

5. Immunization by Injection of the Mixed 3 Antigens into Chicks and Boosting Injection into Egg Laying Hens.

The ratio the antigen and emulsifier injected into the 12 weeks old chicks was E. coli:Salmonella enteritidis:Salmonella typhimurium:emulsifying adjuvant=3.5:1.8:1.7:3. In detail, the first injection were done with 1 ml of the emulsified mixed antigen proteins comprising 0.35 ml antigen of nonliving E. coli 4.0×10⁸/ml, 0.18 ml antigen of nonliving Salmonella enteritidis 4.0×10⁸/ml, and 0.17 ml antigen of nonliving Salmonella typhimurium 4.0×10⁸/ml emulsified with 0.3 ml of aluminum hydroxide. From the second time as boosting injections, the mixed antigen emulsified with emulsifying adjuvant ISA25 were used. Two times of injection with 1 ml of the mixture of antigens with same ratio were done into chicks with two week interval. Then, the egg laying hens grown were injected with 0.5 ml each of emulsified mixed antigen proteins comprising 0.35 ml antigen of nonliving E. coli 2.0×10⁸/ml, 0.18 ml antigen of nonliving Salmonella enteritidis 2.0×10⁸/ml, and 0.17 ml antigen of nonliving Salmonella typhimurium 2.0×10⁸/ml emulsified with 0.3 ml of emulsifying adjuvant ISA25 for two times by 3 month interval as a total of 5 times. As a result, the eggs containing anti-E.coli IgY, anti-Salmonella enteritidis IgY and anti-Salmonella typhimurium IgY, simultaneously, were produced from the egg laying hens immunized with the mixture of antigens emulsified.

As another feature of this invention, the first injection for chicks were done on one leg with the mixed antigen proteins emulsified lmiQi comprising antigen of E. coli, Salmonella enteritidis, and Salmonella typhimurium emulsified with Adjuvant complete Freund's in certain ratio. From the second time as boosting injections, the mixed antigen emulsified with adjuvant incomplete Freund's were used. Two times of injection with 1 ml of the mixture of antigens with same ratio were done into chicks with two week interval. Then, the egg laying hens grown were injected with 0.5 ml each of emulsified mixed antigen proteins for two times by 3 month interval as a total of 5 times. As a result, the eggs containing anti-E.coli IgY, anti-Salmonella enteritidis IgY and anti-Salmonella typhimurium IgY, simultaneously, were produced.

In detail, the ratio of antigen mixture was E. coli:Salmonella enteritidis:Salmonella typhimurium:adjuvant complete Freund's=3.5:1.8:1.7:3. The first injection were done with the mixed antigen proteins comprising 0.35 ml antigen of nonliving E. coli 4.0×10⁸/ml, 0.18 ml antigen of nonliving Salmonella enteritidis 4.0×10⁸/ml, and 0.17 ml antigen of nonliving Salmonella typhimurium 4.0×10⁸/ml emulsified with 0.3 ml of Adjuvant complete Freund's. From the second time as boosting injections, the mixed antigen emulsified with Adjuvant incomplete Freund's were used. Two times of injection with 1 ml were done into chicks with two week interval. Then, the egg laying hens grown were injected on leg with 0.5 ml emulsified mixed antigen proteins comprising 0.35 ml antigen of nonliving E. coli 2.0×10⁸/ml, 0.18 ml antigen of nonliving Salmonella enteritidis 2.0×10⁸/ml, and 0.17 ml antigen of nonliving Salmonella typhimurium 2.0×10⁸/ml emulsified with 0.3 ml of adjuvant incomplete Freund's for two times by 3 month interval as a total of 5 times. Then, The eggs containing anti-E.coli IgY, anti-Salmonella enteritidis IgY and anti-Salmonella typhimurium IgY, simultaneously, were produced.

Another feature of this invention is the production methods of the water-soluble anti-bacteria specific immunoproteins and the specific immunoproteins containing anti-mixed bacteria specific antibodies (IgY) comprising the following steps. The 20 g egg yolk containing anti-E.coli IgY, anti-Salmonella enteritidis IgY and anti-Salmonella typhimurium IgY, simultaneously, produced as above and same amount of alkali ionic water (pH9)(20 ml) were added into some container, stirred and let it sit at 5˜10° C. for 24 hours. The 18 volume (720 ml) of the alkali ionic water (pH10) were added and letting it sit for 48 hours, and the water-soluble proteins were separated. The supernatant were concentrated by the Hollow fiber method in the ultrafiltration system and freeze dried.

6. Separation of Egg Yolk, Isolation of the Egg Yolk Powder, Separation of Immunoprotein, and Measurement of the Titer,

a) Separation of Egg Yolk

The yolk of the eggs produced according to the methods described above were separated and collected to a flask or a bottle.

b) Separation of Egg Yolk Powder

The egg yolk powder was produced by the centrifugation and freeze drying of the separated egg yolk.

c) Separation of Immunoprotein, and Measurement of the Titer,

The method for the separation of immunoprotein was as following, the measurement of the titer were done as the method commonly used.

35 g egg yolk without membrane were collected into 250 ml bottle and stirred with 35 ml alkali ionic water (pH 9). After letting it sit at 5˜10° C. for 24 hours, the alkali ionic water(pH 10), 18 volume (1260 ml) of the supernatant of egg yolk and alkali ionic water(1:1) were added and letting it sit for 48 hours for separation. The supernatant were concentrated by the Hollow fiber method in the ultrafiltraion system and freeze dried.

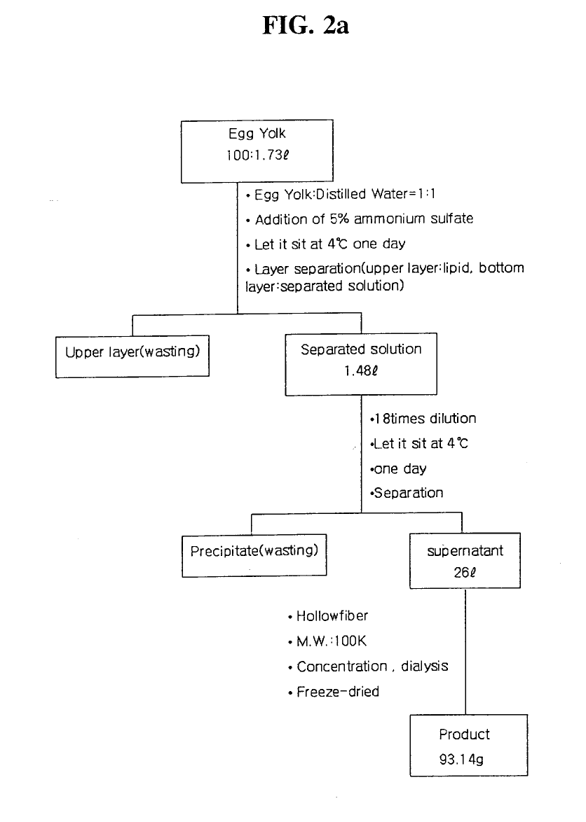

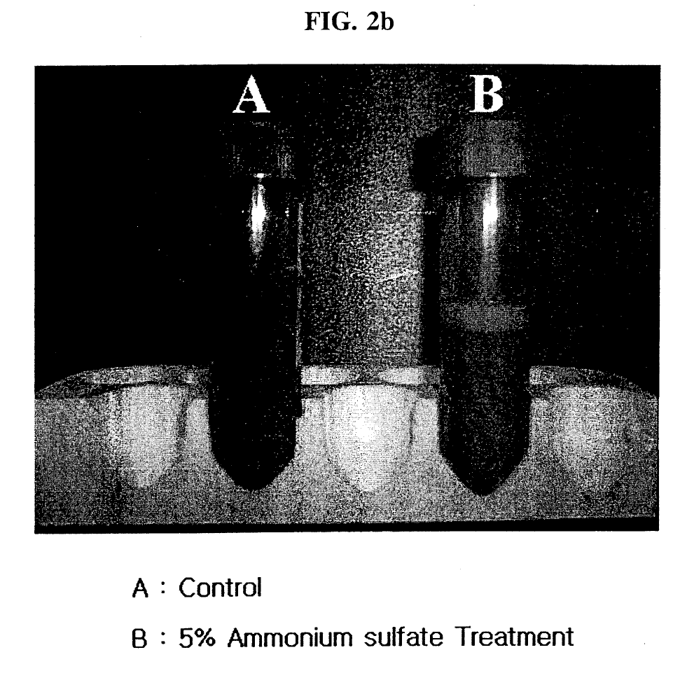

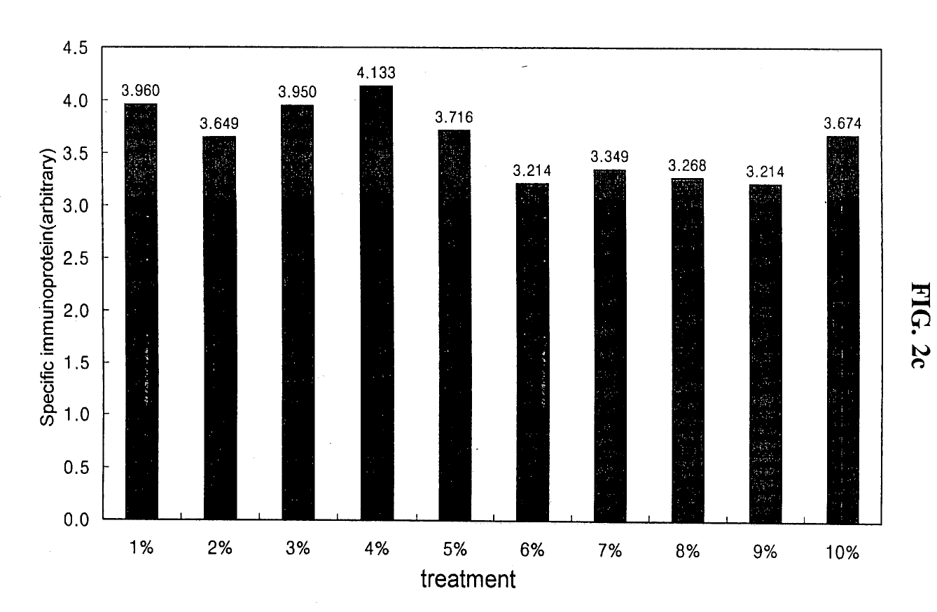

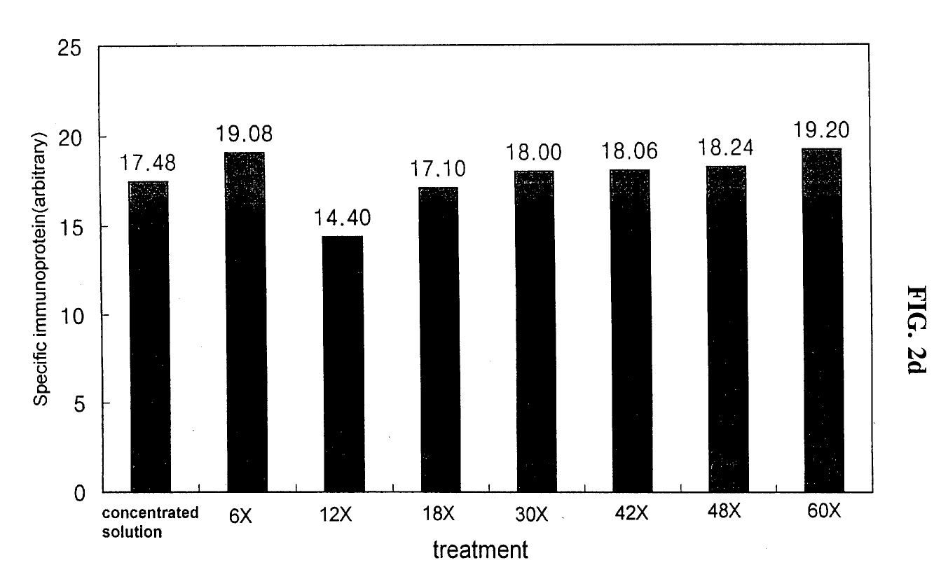

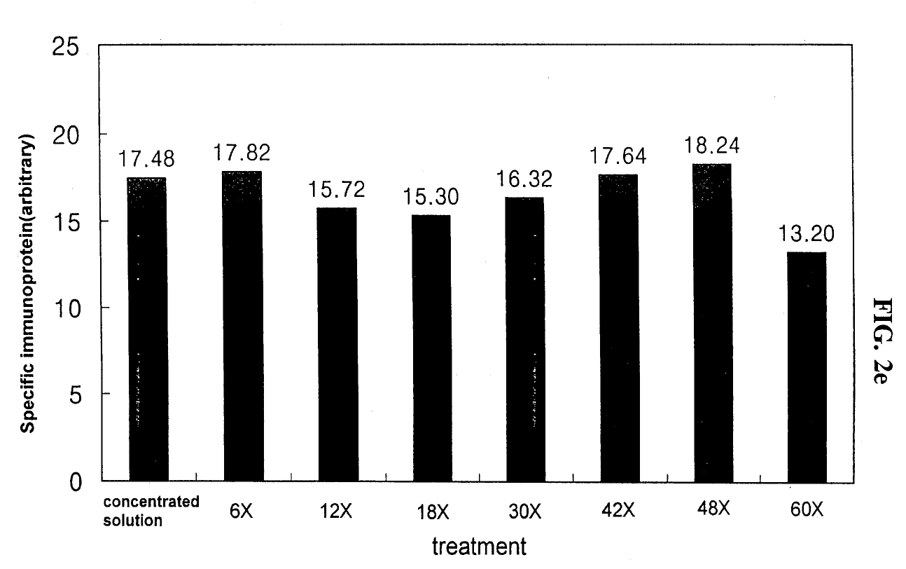

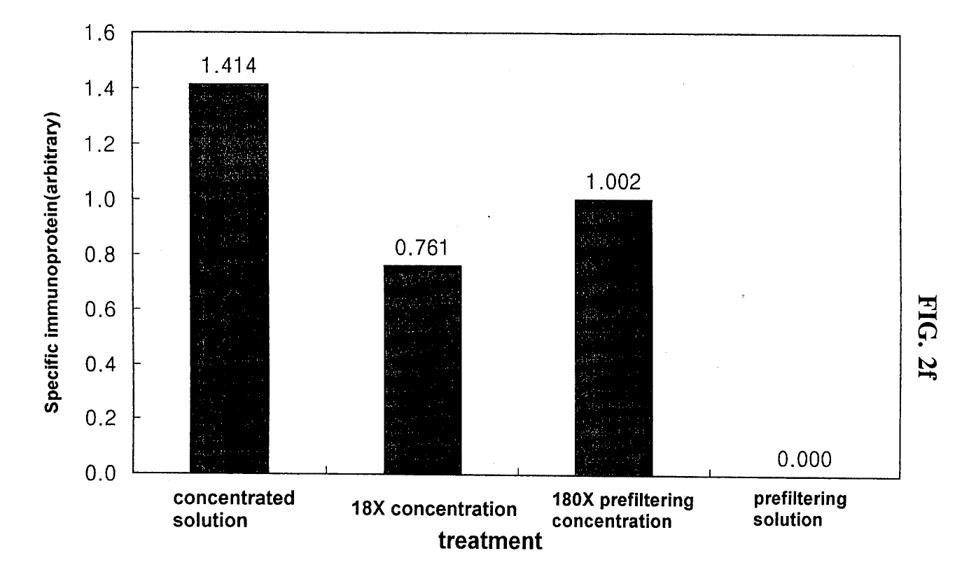

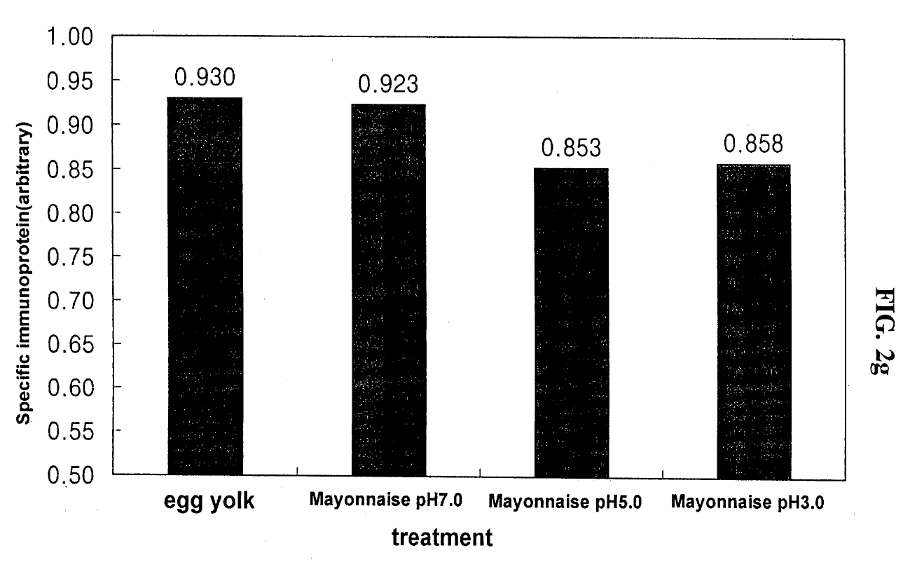

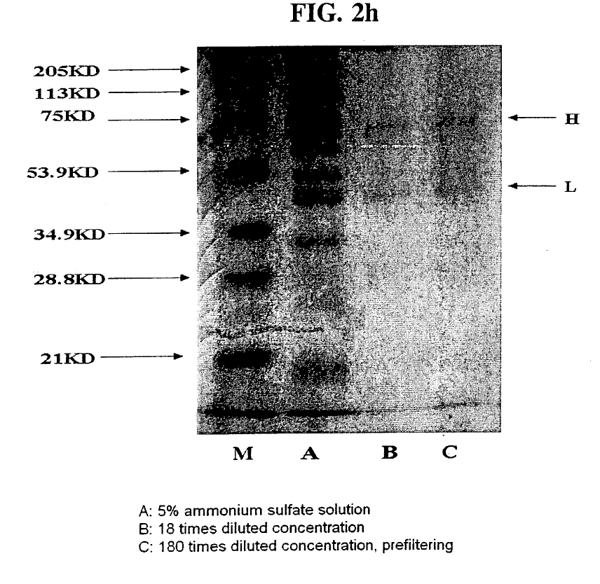

The contents of the specific antibodies in the water-soluble protein, produced by the filtration described above, were measured as following. The contents of the specific antibodies were done by the sandwich ELISA method. Helicobacter pylori were diluted with buffer solution to be O.D. 0.05 at 660 nm. The diluted pathogen was coated into the micro-plate and let it sit overnight. The micro-plate was washed, incubated with the filtered water-soluble protein, washed again, and incubated with 1/10,000 diluted rabbit anti-chick IgG Ab-HRP. TMB was used for the substrate for the HRP, and 2N—H₂SO₄ was used for the reaction stop solution, accordingly. The absorbance at 450 nm were measured (FIGS. 2a, 2b, 2c, 2d).

7. Production of the Yogurt Containing Four Kinds of Antibodies

a) Extraction of the Water-Soluble Specific Immunoprotein

The extraction of the water-soluble specific immunoprotein was done as following.

35 g egg yolk without membrane were collected into 250 ml bottle and stirred with 35 ml alkali ionic water (pH 9). After letting it sit at 5˜10° C. for 24 hours, the alkali ionic water(pH 10), 18 volume (1260 ml) of the supernatant of egg yolk and alkali ionic water were added and letting it sit for 48 hours for separation. The supernatant were concentrated by the Hollow fiber method in the ultrafiltration system and freeze dried.

a) Production of Yogurt Containing Fresh Egg Yolk.

The egg yolk produced by the immunization of the mixed antigens of antigen proteins of Helicobacter pylori, E. coli, Salmonella enteritidis and Salmonella typhimurium were used for the production of the yogurt (table 3, 4). The sterilization problem for the yogurt production is serious. The distribution is limited into the short period due to the process of sterilization at 65° C. for 1 minute, which is inappropriate for the pathogen sterilization. In this invention, the problem of sterilization is solved by the development of the egg containing the anti-Salmonella IgY, which can be used for the yogurt production without sterilization.

b) Production of Yogurt and Ice-Cream Containing the Water-Soluble Specific Immunoprotein Extracted.

The egg yolk produced by the immunization of the mixed antigens of antigen proteins of Helicobacter pylori, E. coli, Salmonella enteritidis and Salmonella typhimurium were separated and mixed with water-soluble protein (the crude IgY). The mixing ratio for the ice-cream and yogurt were given in the Table 1, 2, 3, and 4.

TABLE 1
The example of mixing Ratio for the ice-cream containing
anti-four bacteria specific

| Material | Ratio A | Ratio B | Ratio C |
|---|---|---|---|
| Butter | 5.0% | 5.0% | 8.0% |
| milk | 36.0 | 23.0 | 40.0 |
| whey powder | — | — | 3.0 |
| powdered whole milk | 15.6 | 14.6 | 11.0 |
| powdered skimmed milk | — | — | 4.0 |
| white sugar | 5.0 | 5.0 | 5.5 |
| starch syrup (70% brix) | 14.5 | 13.5 | 14.5 |
| lysozyme | 0.5 | 0.5 | 0.5 |
| stabilizer | 0.6 | 0.6 | 0.6 |
| anti-mixed bacteria*egg yolk powder | 5.0 | — | — |
| anti-mixed bacteria*egg yolk IgY extract | — | — | 0.6 |
| powder | | | |
| anti-mixed bacteria*egg yolk | — | 15.0 | — |
| spicery | some | some | some |
| water | 17.8 | 17.8 | 12.3 |

*mixed bacteria means the egg yolk produced by the immunization with the mixed 4 bacterium

TABLE 2
The example of mixing Ratio for the ice-cream containing
anti-four bacteria

| Material | Ratio A | Ratio B | Ratio C |
|---|---|---|---|
| Cream (milk lipid 35%) | 34.3% | 34.3% | 34.0% |
| skimmed conc. milk (solid 30%) | 25.0 | 10.0 | 25.0 |
| skimmed milk (solid 8%) | 10.0 | 10.0 | 11.0 |
| powdered skimmed milk | 2.0 | 2.0 | 4.0 |
| corn syrup (80% brix) | 5.0 | 5.0 | 5.5 |
| high fructose corn syrup (67% brix) | 14.5 | 14.5 | 14.5 |
| lysozyme | 0.5 | 0.5 | 0.5 |
| stabilizer | 0.6 | 0.6 | 0.6 |
| anti-mixed bacteria*egg yolk powder | 5.0 | — | — |
| anti-mixed bacteria*egg yolk IgY extract | — | — | 0.6 |
| powder | | | |
| anti-mixed bacteria*egg yolk | — | 15.0 | — |
| spicery | some | some | some |
| water | 3.1 | 3.1 | 4.3 |

Mixed bacteria means the egg yolk produced by the immunization with the mixed 4 bacterium

TABLE 3
The example of mixing ratio for the yogurt containing
anti-four bacteria specific immunoproteins

| Material | Ratio A | Ratio B | Ratio C |
|---|---|---|---|
| powdered skimmed milk | 0.40 | 0.40 | 3.05 |
| milk | 96.10 | 93.60 | 95.94 |
| white sugar | 0.60 | 0.60 | 0.34 |
| anti-mixed bacteria*egg yolk powder | 2.50 | — | — |
| anti-mixed bacteria*egg yolk IgY extract | — | — | 0.42 |
| powder | | | |
| anti-mixed bacteria*egg yolk | — | 5.00 | — |
| water | 0.40 | 0.40 | 0.25 |

*mixed bacteria means the egg yolk produced by the immunization with the mixed 4 bacterium

TABLE 4
The example of mixing ratio for the yogurt containing
anti-four bacteria specific immunoproteins

| Material | Ratio A | Ratio B | Ratio C |
|---|---|---|---|
| powdered skimmed milk | 4.40 | 4.40 | 8.05 |
| milk | 78.10 | 80.60 | 75.77 |
| fruit syrup | 14.10 | 9.10 | 15.01 |
| anti-mixed bacteria*egg yolk powder | 2.50 | — | — |
| anti-mixed bacteria*egg yolk IgY extract | — | — | 0.42 |
| powder | | | |
| anti-mixed bacteria*egg yolk | — | 5.00 | — |
| water | 0.40 | 0.40 | 0.25 |
| stabilizer | 0.50 | 0.50 | 0.50 |

*mixed bacteria means the egg yolk produced by the immunization with the mixed 4 bacterium

8. Production of the Yogurt Containing Three Kinds (E. coli, Salmonella enteritidis and Salmonella typhimurium) of Antibodies

a) Extraction of the Water-Soluble Specific Immunoprotein

The extraction of the water-soluble specific immunoprotein were done as following.

35 g egg yolk without membrane were collected into 250 ml bottle and stirred with 35 ml alkali ionic water (pH 9). After letting it sit at 5˜10° C. for 24 hours, the alkali ionic water(pH 10), 18 volume (1260 ml) of the supernatant of egg yolk and alkali ionic water were added and letting it sit for 48 hours for separation. The supernatant were concentrated by the Hollow fiber method in the ultrafiltraion system and freeze dried.

b) Production of Yogurt Containing Fresh Egg Yolk.

The egg yolk produced by the immunization of the mixed antigens of antigen proteins of E. coli, Salmonella enteritidis and Salmonella typhimurium were used for the production of the yogurt (Table 7, 8). The sterilization problem for the yogurt production is serious. The distribution is limited into the short period due to the process of sterilization at 65° C. for 1 minute, which is inappropriate for the pathogen sterilization. In this invention, the problem of sterilization is solved by the development of the egg containing the anti-Salmonella IgY, which can be used for the yogurt production without sterilization.

c) Production of Yogurt and Ice-Cream Containing the Water-Soluble Specific Immunoprotein Extracted.

The egg yolk produced by the immunization of the mixed antigens of antigen proteins of E. coli, Salmonella enteritidis and Salmonella typhimurium were separated and mixed with water-soluble protein (the crude IgY) powder. The mixing ratio for the production of the ice-cream and yogurt were given in the Table 5,6,7,and 8.

TABLE 5
The example of mixing Ratio for the ice-cream containing
anti-three bacteria specific immunoproteins

| Material | Ratio A | Ratio B | Ratio C |
|---|---|---|---|
| Butter | 5.0% | 5.0% | 8.0% |
| milk | 36.0 | 23.0 | 40.0 |
| whey powder | — | — | 3.0 |
| powdered whole milk | 15.6 | 14.6 | 11.0 |
| powdered skimmed milk | — | — | 4.0 |
| white sugar | 5.0 | 5.0 | 5.5 |
| starch syrup (70% brix) | 14.5 | 13.5 | 14.5 |
| lysozyme | 0.5 | 0.5 | 0.5 |
| stabilizer | 0.6 | 0.6 | 0.6 |
| anti-mixed bacteria*egg yolk powder | 5.0 | — | — |
| anti-mixed bacteria*egg yolk IgY extract | — | — | 0.6 |
| powder | | | |
| anti-mixed bacteria*egg yolk | — | 15.0 | — |
| spicery | some | some | some |
| water | 17.8 | 17.8 | 12.3 |

*mixed bacteria means the egg yolk produced by the immunization with the mixed 3 bacterium

TABLE 6
The example of mixing Ratio for the icecream containing
anti-three bacteria specific immunoproteins

| Material | Ratio A | Ratio B | Ratio C |
|---|---|---|---|
| cream (milk fat 35%) | 34.3% | 34.3% | 34.0% |
| skimmed conc. milk (solid 30%) | 25.0 | 10.0 | 25.0 |
| skimmed milk (solid 8%) | 10.0 | 10.0 | 11.0 |
| powdered skimmed milk | 2.0 | 2.0 | 4.0 |
| corn syrup (80% brix) | 5.0 | 5.0 | 5.5 |
| high fructose corn syrup (67% brix) | 14.5 | 14.5 | 14.5 |
| lysozyme | 0.5 | 0.5 | 0.5 |
| stabilizer | 0.6 | 0.6 | 0.6 |
| anti-mixed bacteria*egg yolk powder | 5.0 | — | — |
| anti-mixed bacteria*egg yolk IgY extract | — | — | 0.6 |
| powder | | | |
| anti-mixed bacteria*egg yolk | — | 15.0 | — |
| spicery | some | some | some |
| water | 3.1 | 3.1 | 4.3 |

*mixed bacteria means the egg yolk produced by the immunization with the mixed 3 bacterium

TABLE 7
The example of mixing Ratio for the yogurt containing
anti-three bacteria specific immunoproteins

| Material | Ratio A | Ratio B | Ratio C |
|---|---|---|---|
| powdered skimmed milk | 0.40 | 0.40 | 3.05 |
| milk | 96.10 | 93.60 | 95.94 |
| white sugar | 0.60 | 0.60 | 0.34 |
| anti-mixed bacteria*egg yolk powder | 2.50 | — | — |
| anti-mixed bacteria*egg yolk IgY extract | — | — | 0.42 |
| powder | | | |
| anti-mixed bacteria*egg yolk | — | 5.00 | — |
| water | 0.40 | 0.40 | 0.25 |

*mixed bacteria means the egg yolk produced by the immunization with the mixed 3 bacterium

TABLE 8
The example of mixing Ratio for the yogurt containing
anti-three bacteria specific immunoprotein

| Material | Ratio A | Ratio B | Ratio C |
|---|---|---|---|
| powdered skimmed milk | 4.40 | 4.40 | 8.05 |
| milk | 78.10 | 80.60 | 75.77 |
| fruit syrup | 14.10 | 9.10 | 15.01 |
| anti-mixed bacteria*egg yolk powder | 2.50 | — | — |
| anti-mixed bacteria*egg yolk IgY extract | — | — | 0.42 |
| powder | | | |
| anti-mixed bacteria*egg yolk | — | 5.00 | — |
| water | 0.40 | 0.40 | 0.25 |
| stabilizer | 0.50 | 0.50 | 0.50 |

mixed bacteria means the egg yolk produced by the immunization with the mixed 3 bacterium

### Example 2

Example 2 is about the mixture of the specific immunoproteins produced by mixing each antibody made separately, and the yogurt and icecream containing the specific immunoproteins extracted from the eggs produced by the same method.

1. Isolation and Identification of Enteropathogenic E. coli and Antigen Production

The enteropathogenic E. coli used in this invention is isolated from human. Isolation and identification of enteropathogenic E. coli (ETEC) and antibody production were done as described in Example 1.

2. Production of the Helicobacter pylori Antigen.

The production of the Helicobacter pylori antigen were done as described in Example 1.

3. Production of the Salmonella enteritidis Antigen, and Salmonella typhimurium. Antigen.

The production of the Salmonella enteritidis antigen, and Salmonella typhimurium antigen was done as described in Example 1.

4. Immunization by Injection of the 4 Antigens into Chicks Separately and Boosting Injection into Egg Laying Hens.

a) 12 weeks old chicks were immunized with 1 ml (10⁸/ml) of each of E. coli, Helicobacter pylori, Salmonella enteritidis and Salmonella typhimurium, emulsified with emulsifying adjuvant in a 1:1 ratio separately, which comprise the 0.5 ml antigen of nonliving E. coli 2.0×10⁸/ml and the 0.5 ml of aluminum hydroxide, 0.5 ml antigen of nonliving Salmonella enteritidis 2.0×10⁸/ml and the 0.5 ml of aluminum hydroxide, 0.5 ml antigen of nonliving Salmonella typhimurium 2.0×10⁸/ml and the 0.5 ml of aluminum hydroxide, and 0.5 ml antigen of nonliving Helicobacter pylori 2.0×10⁸/ml and the 0.5 ml of aluminum hydroxide in the 1:1 ratio accordingly. From the second weeks, 1 ml each of the antigen proteins emulsified with the emulsifying adjuvant ISA25 for two times by 2 weeks interval. The grown egg laying hens were immunized with 0.5 ml each of antigen proteins emulsified, which comprise the 0.5 ml antigen of nonliving E. coli 2.0×10⁸/ml and the 0.5 ml of emulsifying adjuvant ISA25, 0.5 ml antigen of nonliving Salmonella enteritidis 2.0×10⁸/ml and the 0.5 ml of emulsifying adjuvant ISA25, 0.5 ml antigen of nonliving Salmonella typhimurium 2.0×10⁸/ml and the 0.5 ml of emulsifying adjuvant ISA25, and 0.5 ml antigen of nonliving Helicobacter pylori 2.0×10⁸/ml and the 0.5 ml of emulsifying adjuvant ISA25 in the 1:1 ratio accordingly for two times by 3 month interval. The four antigens (E. coli, Helicobacter pylori, Salmonella enteritidis and Salmonella typhimurium) were not mixed and injected separately, as a total of 5 times.

b) 12 weeks old chicks were immunized with 1 ml (10⁸/ml) of each of E. coli, Helicobacter pylori, Salmonella enteritidis and Salmonella typhimurium, emulsified with emulsifying adjuvant in a 1:1 ratio separately, which comprise the 0.5 ml antigen of nonliving E. coli 2.0×10⁸/ml and the 0.5 ml of Adjuvant complete Freund's, 0.5 ml antigen of nonliving Salmonella enteritidis 2.0×10⁸/ml and the 0.5 ml of Adjuvant complete Freund's, 0.5 ml antigen of nonliving Salmonella typhimurium 2.0×10⁸/ml and the 0.5 ml of Adjuvant complete Freund's, and 0.5 ml antigen of nonliving Helicobacter pylori 2.0×10⁸/ml and the 0.5 ml of Adjuvant complete Freund's in the 1:1 ratio accordingly. From the second weeks, 1 ml each of the antigen proteins emulsified with the Adjuvant incomplete Freund's for two times by 2 weeks interval. The grown egg laying hens were immunized with 0.5 ml each of emulsified antigen proteins, which comprise the 0.5 ml antigen of nonliving E. coli 2.0×10⁸/ml and the 0.5 ml of Adjuvant incomplete Freund's, the antigen proteins 0.5 ml antigen of nonliving Salmonella enteritidis 2.0×108/ml and the 0.5 ml of Adjuvant incomplete Freund's, 0.5 ml antigen of nonliving Salmonella typhimurium 2.0×10⁸/ml and the 0.5 ml of Adjuvant incomplete Freund's, and 0.5 ml antigen of nonliving Helicobacter pylori 2.0×10⁸/ml and the 0.5 ml of Adjuvant incomplete Freund's in the 1:1 ratio accordingly for two times by 3 month interval. The four antigens (E. coli, Helicobacter pylori, Salmonella enteritidis and Salmonella typhimurium) were not mixed and injected separately, as a total of 5 times. As a result, The eggs containing anti-E.coli IgY, anti-Salmonella enteritidis IgY and anti-Salmonella typhimurium IgY, separately, were produced from the egg-laying hens immunized with the emulsified separate antigens.

5. Separation of Egg Yolk, Isolation of the Egg Yolk Powder, Separation of Immunoprotein, and Measurement of the Titer,

The separation of egg yolk, isolation of the egg yolk powder, separation of immunoprotein, and measurement of the titer were done as example 1.

6. Production of the Yogurt and Icecream Containing Mixed Immunoproteins.

As described above, the water-soluble specific immunoprotein powder (crude IgY), fresh egg yolk, and dried egg yolk were used as food additives in the ratios of 1:1:1:1, 2:1:1:1, 3:1:1:1, 4:1:1:1, and 5:1:1:1 (Helicobacter pylori, E. coli, Salmonella enteritidis and Salmonella typhimurium), and the mixing ratio for the icecream and yogurt were given in the Table 9, 10, 11, and 12.

TABLE 9
The example of the mixing ratio for the icecream

| Material | Ratio A | Ratio B | Ratio C |
|---|---|---|---|
| Butter | 5.0% | 5.0% | 8.0% |
| milk | 36.0 | 31.0 | 40.0 |
| whey powder | — | — | 3.0 |
| powdered whole milk | 15.6 | 14.6 | 11.0 |
| powdered skimmed milk | — | — | 4.0 |
| white sugar | 5.0 | 5.0 | 5.5 |
| starch syrup (70% brix) | 14.5 | 13.5 | 14.5 |
| stabilizer | 0.6 | 0.6 | 0.6 |
| anti-mixed bacteria*egg yolk powder | 5.5 | — | — |
| anti-mixed bacteria*egg yolk IgY | — | — | 1.1 |
| extract powder | | | |
| anti-mixed bacteria*egg yolk | — | 12.5 | — |
| spicery | some | some | some |
| water | 17.8 | 17.8 | 12.3 |

*mixed bacteria means the mixture of the four kinds of dried (or fresh) egg yolk produced separately.

TABLE 10
The example of the mixing ratio for the icecream

| Material | Ratio A | Ratio B | Ratio C |
|---|---|---|---|
| cream (lipid 35%) | 34.3% | 34.3% | 34.0% |
| skimmed conc. milk (solid 30%) | 25.5 | 20.5 | 25.0 |
| skimmed milk (solid 8%) | 10.0 | 10.0 | 11.0 |
| powdered skimmed milk | 2.0 | 2.0 | 4.0 |
| corn syrup (80% brix) | 5.0 | 5.0 | 5.5 |
| high fluctose corn syrup (67% brix) | 14.5 | 14.5 | 14.5 |
| stabilizer | 0.6 | 0.6 | 0.6 |
| anti-mixed bacteria*egg yolk powder | 5.0 | — | — |
| anti-mixed bacteria*egg yolk IgY | — | — | 0.6 |
| extract powder | | | |
| anti-mixed bacteria*egg yolk | — | 10.0 | — |
| spicery | some | some | some |
| water | 3.1 | 3.1 | 4.8 |

*mixed bacteria means the mixture of the four kinds of dried (or fresh egg yolk) produced separately.

TABLE 11
The example of the mixing ratio for the yogurt

| Material | Ratio A | Ratio B | Ratio C |
|---|---|---|---|
| powdered skimmed milk | 0.40 | 0.40 | 3.05 |
| milk | 95.50 | 93.05 | 95.62 |
| white sugar | 0.60 | 0.60 | 0.10 |
| anti-mixed bacteria* egg | 2.55 | — | — |
| yolk powder | | | |
| anti-mixed bacteria* egg | — | — | 0.43 |
| yolk IgY extract powder | | | |
| anti-mixed bacteria* egg yolk | — | 5.00 | — |
| water | 0.40 | 0.40 | 0.25 |
| stabilizer | 0.55 | 0.55 | 0.55 |

*mixed bacteria means the mixture of the four kinds of dried (or fresh egg yolk) produced separately.

TABLE 12
The example of the mixing ratio for the yogurt

| Material | Ratio A | Ratio B | Ratio C |
|---|---|---|---|
| powdered skimmed milk | 4.40 | 4.40 | 8.10 |
| milk | 78.05 | 80.60 | 75.63 |
| fruit syrup | 14.10 | 9.10 | 15.01 |
| anti-mixed bacteria* egg | 2.55 | — | — |
| yolk powder | | | |
| anti-mixed bacteria* egg | — | — | 0.51 |
| yolk IgY extract powder | | | |
| anti-mixed bacteria* egg yolk | — | 5.0 | — |
| water | 0.40 | 0.40 | 0.25 |
| stabilizer | 0.50 | 0.50 | 0.50 |

*mixed bacteria means the mixture of the four kinds of dried (or fresh egg yolk) produced separately.

### Example 3

The experimental methods of this example were given in the FIG. 2a.

The detailed description were as following. To separate the egg yolk containing the IgY, lipid and lipoprotein, the egg yolk were diluted with the same amount of distilled water. Ten treatments of the diluted egg yolk were prepared and stirring 1%˜10% ammonium sulfate were slowly added into the treatments with stirring for complete melting. After incubating ten of treatments at 5° C. one day, the upper layer and bottom layer were separated according to the concentration difference. Some of the lipids and proteins were floated in the upper layer, the ignorable amount of the precipitation can be seen in the bottom layer. (FIG. 2b)

As seen in the FIG. 2c, in the treatment added with 1% ammonium sulfate, no separation and a little amount of the precipitation were seen, similar to the treatment added with 2% ammonium sulfate. In the treatment added with 3% ammonium sulfate, the lipid layer is seen in the upper layer, similar precipitation as seen in 1% treatment were observed. In the treatment added with 4% ammonium sulfate, a little more separation of lipid layer and precipitation were seen. In the treatment added with 5%˜6% ammonium sulfate, the thick lipid layer were formed in the upper layer, and a little amount of precipitation were seen. The result of the treatment added with 7˜9% ammonium sulfate were pretty good, but those of the treatment added with 10% ammonium sulfate were not as good as those of the 7˜9% treatment. In other word, the best condition for the lipid removal was adding the 5˜6% ammonium sulfate. In terms of the titer of IgY, the treatment added with 4% ammonium sulfate gave the best result for the titer of IgY, as seen in the FIG. 2c. The treatment added with 5% ammonium sulfate, shows the thick lipid layer was best for the lipid removal. The solution without lipid can be obtained by collecting the bottom layer from the bottom.

Experimental Result

For the separation methods of the protein utilizing ammonium sulfate (Mathews, 1990) as commonly used prior arts, higher concentration of ammonium sulfate utilized, more precipitation of the proteins were observed, which require the purification of the precipitant. Often, the precipitant in the treatments added with the 10%˜20% ammonium sulfate were discarded after centrifugation to get highly purified protein, and the protein precipitated after adding 20% ammonium sulfate were used.

This invention utilized the floating characteristics of the lipid and the coagulating characteristics by means of ammonium sulfate to separate the lipid of the egg yolk, the lipoprotein and specific immunoproteins, which is hard to isolate without using the many solvent and precipitation inducing agent.

### Example 4

To remove the lipid, water-soluble protein and pigment of egg yolk remained in the solution separated from lipid, the following experiment was done.

The seven treatments were diluted with distilled water by the factor of ×6, ×12, ×18, ×30, ×42, ×48, ×60, and incubated at 5° C. one day. The supernatant were separated carefully without precipitant. While the ×6 diluted treatment contained precipitants, the yellow color of egg yolk was remained in the supernatant, and the precipitant were soon mixed with supernatant. The ×12, ×18, ×30 diluted treatment contained precipitants not mixed with supernatant easily. The ×42, ×48, ×60 diluted treatment contained precipitants but mixed with supernatant easily, Therefore, the appropriate dilution factor for precipitation were ×12, ×18, of which precipitant were so sticky that tap water was used to wash out. At the same time, the titer of the IgY of the supernatant were measured the titer yield of the IgY of the diluents by the factor of 6 and 60 were 109%, 110%, higher than standard. Therefore, the titer of the IgY became higher in the diluents were increased by the effect of the water-soluble immunoprotein. The IgY titer of other diluted treatments were over 100% except ×12, ×18 diluted treatments. The dilution factor 18 were selected, since it cause no melting of precipitant and complete removal of the pigment.

Since it is suggested by Mathews, 1990 and Stryer, 1998, that homogenization by the blender and the homogenizer is good for homogenization for the purpose of the destructing the cells, because of being quick and low in protein degradation by the protease, the homogenizer was utilized to destruct the cells and tissues, and diluted by the factor of ×6, ×12, ×18, ×30, ×42,×48, ×60, incubated at 5° C. overnight, and repeated the same experiment. For the separation process, the precipitant and supernatant were all mixed, came out together, and resulted the data shown at the Table 14 and FIG. 2e. Since no homogeniation were better in terms of the tier of IgY, the decision not using blender or homogenizer was made in regard of process and economics.

TABLE 13
The potency change of the specific immunoprotein IgY according to dilution factor

| Dilution factor | | | | | | | | |
|---|---|---|---|---|---|---|---|---|
| (no homogenization) | | | | | | | | |
| homogenization) | Before dilution | X6 | X12 | X18 | X30 | X42 | X48 | X60 |
| Potency | 17.48 | 19.08 | 14.40 | 17.10 | 18.00 | 18.06 | 18.24 | 19.20 |

TABLE 14
The potency change of the specific immunoprotein IgY according to
homogenization

| Dilution factor | | | | | | | | |
|---|---|---|---|---|---|---|---|---|
| (homogenization) | Before dilution | X6 | X12 | X18 | X24 | X36 | X48 | X60 |
| potency | 17.48 | 17.82 | 15.72 | 15.30 | 16.32 | 17.64 | 18.24 | 13.20 |

While the Akida, 1993 utilized the dilution methods utilizing distilled water, which involves the addition of dextran, xanthan gum, PEG(Poly Ethylene Glycol), ethanol, sodium sulfate as a precipitation inducing agent after dilution and the centrifugation for separation, this invention utilized small amount of the ammonium sulfate to coagulate some lipid and protein in the upper layer for the removal of the lipid, which is the opposite of the separation method of protein utilizing the ammonium sulfate. The prior art utilized ammonium sulfate for precipitation, different from this invention which utilized ammonium sulfate for floating and coagulating. In this invention, 5% ammonium sulfate, egg yolk, and distilled water (1:1) was used to separate the lipid and protein by coagulating in group due to the gravity difference, followed by the coagulating-precipitation of the egg yolk pigment and water-soluble protein by means of the diluting with distilled water.

Experiment of the product produced according to this invention.

To produce the product containing high content of IgY, the separated supernatant were diluted ×18 and concentrated by the concentrator with amicon-2000 hollow filter: M.W 100K, HIP100-43. Additionally, some part of the same concentrated supernatant were filtered and concentrated by TOYO filter paper No.2, 2˜3 layer, and the concentrated solution were diluted with 10 volumes of distilled water, and dialyzed for concentration.

The titer of the IgY in treatments produced by these two methods described above and freeze-dried, were given in the FIG. 2e. The yield of the product produced by the steps comprising diluting ×18, concentrating, diluting 10 times, concentrating and dialyzing were 70.8%, which is higher than the yield of the products (yield 53.8%) produced by the steps comprising diluting ×18 and concentrating, and also the purity of those were better than the other due to better purification.

The titer of the IgY of the mayonnaise produced.

As a experimentation, the mayonnaise were produced from the egg yolk produced in this invention by employing the condition of pH3, pH5, pH7 from the egg yolk produced in this invention. The yield of the IgY employing the condition of pH7 was 92.3% , best of all, as given in the FIG. 2g, and the yield of the pH3 and pH5 was 85.8%, and 85.3%, individually. Therefore, it can be learned that there was no loss of the IgY titer due to the production of the mayonnaise using the egg yolk produced in this invention.

The purification test of the product produced in this invention

The supernatant separated and freeze-dried were white, the purification comparison of the freeze-dried product before purification and after purification by SDS-PAGE was given in FIG. 2h. As seen in FIG. 8, the product produced in this invention were much more purified than others. It has been reported that not using centrifugation resulted in low yield, no use of the precipitation inducing agent cause hard on purification, which lead to the decrease in the IgY titer of the supernatant separated. But the product produced in the invention were superior in terms of the yield and purity, which show that this invention was advantageous for saving the cost and time of production, and the mass production.

The invention is not limited by the detailed description of the preferred embodiments and the figures described above. The common person in this technical field can enforce this invention as the variety of the modified form, and the modification should be included in the claims described.

What is claimed is:

1. The method for producing the egg containing anti-mixed bacteria specific immunoproteins; anti-E.coli IgY and anti-Helicobacter pylori IgY and anti-Salmonella enteritidis IgY and anti-Salmonella typhimurium IgY simultaneously, comprising the steps of:
immunizing chicks by administering with 1 ml of mixed antigen proteins of E. coli, Helicobacter pylori, Salmonella enteritidis and Salmonella typhimurium, emulsified with aluminum hydroxide in a certain ratio at a first time, and 1 ml each of emulsified mixed antigen proteins using the emulsifying adjuvant ISA25 for two times by 1 week interval,
and immunizing the grown egg laying hens with 0.5 ml each of emulsified mixed antigen proteins using the emulsifying adjuvant ISA25 in the same ratio for two times by 3 month interval as a total of 5 times.

2. The method for producing the egg, according to claim 1, containing anti-mixed bacteria specific immunoproteins; anti-E.coli IgY and anti-Helicobacter pylori IgY and anti-Salmonella enteritidis IgY and anti-Salmonella typhimurium IgY simultaneously, comprising the steps of:
administering chicks with 1 ml of the mixed antigen proteins of E. coli, Helicobacter pylori, Salmonella enteritidis and Salmonella typhimurium, emulsified with aluminum hydroxide in a certain ratio at a first time, which comprise 0.15 ml antigen of nonliving E. coli 4.0×10⁸/ml, 0.10 ml antigen of nonliving Salmonella enteritidis 4.0×10⁸/ml, 0.10 ml antigen of nonliving Salmonella typhimurium 4.0×10⁸/ml, and 0.35 ml antigen of nonliving Helicobater pylori 4.0×10⁸/ml, emulsified with 0.3 ml of aluminum hydroxide in the ratio of E. coli:Salmonella enteritidis:Salmonella typhimurium:Helicobacter pylori:emulsifying adjuvant=1.5:1:1:3.5:3 and for second and third injection, 1 ml of mixed antigen proteins diluted with the emulsifying adjuvant ISA25 in 1:1 ratio for two times by 1 week interval,
and immunizing the grown egg laying hens with 0.5 ml each of emulsified mixed antigen proteins comprising 0.15 ml antigen of nonliving E. coli 2.0×10⁸/ml, 0.10 ml antigen of nonliving Salmonella enteritidis 2.0×10⁸/ml, 0.10 ml antigen of nonliving Salmonella typhimurium 2.0×10⁸/ml, and 0.35 ml antigen of nonliving Helicobacter pylori 2.0×10⁸/ml, emulsified with 0.3 ml of emulsifying adjuvant ISA25 in the same ratio for two times by 3 month interval as a total of 5 times.

3. The method for producing the egg containing anti-mixed bacteria specific immunoproteins; anti-E.coli IgY and anti-Helicobacter pylori IgY and anti-Salmonella enteritidis IgY and anti-Salmonella typhimurium IgY simultaneously, comprising the steps of:
immunizing chicks by administering with 1 ml of mixed antigen proteins of E. coli, Helicobacter pylori, Salmonella enteritidis and Salmonella typhimurium, emulsified with Adjuvant complete Freund's in a certain ratio at a first time, and 1 ml each of emulsified mixed antigen proteins using Adjuvant incomplete Freund's for two times by 1 week interval, again immunizing the grown egg laying hens with 0.5 ml each of the emulsified mixed antigen proteins in the same ratio for two times by 3 month interval as a total of 5 times.

4. The method for producing the egg, according to claim 3, containing anti-mixed bacteria specific immunoproteins; anti-E.coli IgY and anti-Helicobacter pylori IgY and anti-Salmonella enteritidis IgY and anti-Salmonella typhimurium IgY simultaneously, comprising the steps of:
administering chicks with 1 ml of the mixed antigen proteins of E. coli, Helicobacter pylori, Salmonella enteritidis and Salmonella typhimurium, emulsified with Adjuvant complete Freund's in a certain ratio at a first time, which comprise 0.15 ml antigen of nonliving E. coli 4.0×10⁸/ml, 0.10 ml antigen of nonliving Salmonella enteritidis 4.0×10⁸/ml, 0.10 ml antigen of nonliving Salmonella typhimurium 4.0×10⁸/ml, and 0.35 ml antigen of nonliving Helicobacter pylori 4.0×10⁸/ml, emulsified with 0.3 ml of Adjuvant incomplete Freund's in the ratio of E. coli:Salmonella enteritidis:Salmonella typhimurium:Helicobacter pylori:emulsifying adjuvant=1.5:1:1:3.5:3 and for second and third injection, 1 ml of mixed antigen proteins diluted with the Adjuvant incomplete Freund's in 1:1 ratio for two times by 1 week interval,
and immunizing the grown egg laying hens with 0.5 ml each of emulsified mixed antigen proteins comprising 0.15 ml antigen of nonliving E. coli 2.0×10⁸/ml, 0.10 ml antigen of nonliving Salmonella enteritidis 2.0×10⁸/ml, 0.10 ml antigen of nonliving Salmonella typhimurium 2.0×10⁸/ml, and 0.35 ml antigen of nonliving Helicobacter pylori 2.0×10⁸/ml, emulsified with 0.3 ml of Adjuvant incomplete Freund's in the same ratio for two times by 3 month interval as a total of 5 times.

5. The method for producing the egg containing anti-mixed bacteria specific immunoproteins; anti-E.coli IgY, anti-Salmonella enteritidis IgY and anti-Salmonella typhimurium IgY simultaneously, comprising the steps of:
immunizing chicks by administering with 1 ml of mixed antigen proteins of E. coli, Salmonella enteritidis and Salmonella typhimurium, emulsified with aluminium hydroxide in a certain ratio at a first time, and 1 ml each of emulsified mixed antigen proteins using the emulsifying adjuvant ISA25 for two times by 1 week interval,
and immunizing the grown egg laying hens with 0.5 ml each of emulsified mixed antigen proteins using the emulsifying adjuvant ISA25 in the same ratio for two times by 3 month interval as a total of 5 times

6. The method for producing the egg, according to claim 5, comprising the steps of:
administering chicks with 1 ml of the mixed antigen proteins of E. coli, Salmonella enteritidis and Salmonella typhimurium, emulsifier in the ratio of 3.5:1.8:1.7:3, which comprise 0.35 ml antigen of nonliving E. coli 4.0×10⁸/ml, 0.18 ml antigen of nonliving Salmonella enteritidis 4.0×10⁸/ml, 0.17 ml antigen of nonliving Salmonella typhimurium 4.0×10⁸/ml, emulsified with 0.3 ml of aluminium hydroxide, and for second and third injection, 1 ml of mixed antigen proteins diluted with the emulsifying adjuvant ISA25 in 1:1 ratio for two times by 1 week interval,
and immunizing the grown egg laying hens with 0.5 ml each of emulsified mixed antigen proteins comprising 0.35 ml antigen of nonliving E. coli 2.0×10⁸/ml, 0.18 ml antigen of nonliving Salmonella enteritidis 2.0×10⁸/ml, 0.17 ml antigen of nonliving Salmonella typhimurium 2.0×10⁸/ml, emulsified with 0.3 ml of emulsifying adjuvant ISA25, in the same ratio for two times by 3 month interval as a total of 5 times.

7. The method for producing the egg containing anti-mixed bacteria specific immunoproteins; anti-E.coli IgY, anti-Salmonella enteritidis IgY and anti-Salmonella typhimurium IgY simultaneously, comprising the steps of:
immunizing chicks by administering with 1 ml of mixed antigen proteins of E. coli, Salmonella enteritidis and Salmonella typhimurium, emulsified with Adjuvant complete Freund's in a certain ratio at a first time, and 1 ml each of emulsified mixed antigen proteins using Adjuvant incomplete Freund's for two times by 1 week interval,
and immunizing the grown egg laying hens with 0.5 ml each of emulsified mixed antigen proteins using Adjuvant incomplete Freund's in the same ratio for two times by 3 month interval as a total of 5 times

8. The method for producing the egg, according to claim 7, comprising the steps of:
administering chicks with 1 ml of the mixed antigen proteins of E. coli, Salmonella enteritidis and Salmonella typhimurium, Adjuvant incomplete Freund's in the ratio of 3.5:1.8:1.7:3, which comprise 0.35 ml antigen of nonliving E. coli 4.0×10⁸/ml, 0.18 ml of nonliving Salmonella enteritidis 4.0×10⁸/ml, 0.17 ml antigen of nonliving Salmonella typhimurium 4.0×10⁸/ml, emulsified with 0.3 ml of Adjuvant complete Freund's, and for second and third injection, 1 ml of mixed antigen proteins diluted with the emulsifying Adjuvant incomplete Freund's in 1:1 ratio for two times by 1 week interval,
and immunizing the grown egg laying hens with 0.5 ml each of emulsified mixed antigen proteins comprising 0.35 ml antigen of nonliving E. coli 2.0×10⁸/ml, 0.18 ml antigen of nonliving Salmonella enteritidis 2.0×10⁸/ml, 0.17 ml antigen of nonliving Salmonella typhimurium 2.0×10⁸/ml, emulsified with 0.3 ml of Adjuvant incomplete Freund's, in the same ratio for two times by 3 month interval as a total of 5 times.

9. The eggs containing anti-mixed bacteria specific immunoproteins produced according to the methods in claims 1-8

10. The method for producing anti-mixed bacteria specific immunoproteins, comprising the steps of:
collecting 35 g egg yolk of the egg containing anti-mixed bacteria specific immunoproteins into 250 ml bottle, agitating in 35 ml alkali ionic water (pH 9), incubating at 5˜10° C. for 24 hours, adding the alkali ionic water (18 volume (1260 ml) of the supernatant of egg yolk and alkali ionic water), incubating for 48 hours for separation, concentrating the supernatant by the Hollow fiber method in the ultrafiltraion system and freeze-drying.

11. Anti-mixed bacteria specific immunoprotein produced according to the methods in claim 10.

12. The mixed composition of anti-mixed bacteria specific immunoprotein powder composed of the water-soluble specific immunoproteins(crude IgY)(E. coli:Salmonella enteritidis:Salmonella typhimurium:Helicobacter pylori) isolated from the eggs containing specific immunoproteins separately, produced by the methods comprising the steps of;
immunizing chicks by administering with 1 ml (10⁸/ml) antigen proteins of E. coli, Helicobacter pylori, Salmonella enteritidis and Salmonella typhimurium each, emulsified with emulsifying adjuvant in a 1:1 ratio separately at a first time, which comprise 0.5 ml antigen of nonliving E. coli 2.0×10⁸/ml and the 0.5 ml of Adjuvant complete Freund's, 0.5 ml antigen of nonliving Salmonella enteritidis 2.0×10⁸/ml and the 0.5 ml of Adjuvant complete Freund's, 0.5 ml antigen of nonliving Salmonella typhimurium 2.0×10⁸/ml and 0.5 ml of Adjuvant complete Freund's, and 0.5 ml antigen of nonliving Helicobacter pylori 2.0×10⁸/ml and the 0.5 ml of Adjuvant complete Freund's accordingly and 1 ml each of the antigen proteins emulsified with the Adjuvant incomplete Freund's for two times by 2 weeks interval,
and immunizing the grown egg laying hens with 0.5 ml each of emulsified antigen proteins, which comprise 0.5 ml antigen of nonliving E. coli 2.0×10⁸/ml and 0.5 ml of Adjuvant incomplete Freund's, 0.5 ml antigen of nonliving Salmonella enteritidis 2.0×10⁸/ml and 0.5 ml of Adjuvant incomplete Freund's, 0.5 ml antigen of nonliving Salmonella typhimurium 2.0×10⁸/ml and 0.5 ml of Adjuvant incomplete Freund's, and 0.5 ml antigen of nonliving Helicobacter pylori 2.0×10⁸/ml and 0.5 ml of Adjuvant incomplete Freund's, accordingly, for two times by 3 month interval as a total of 5 times.

13. The mixed composition of anti-mixed bacteria specific immunoprotein powder composed of the water-soluble specific immunoproteins(crude IgY)(E. coli:Salmonella enteritidis:Salmonella typhimurium:Helicobacter pylori) isolated from the eggs containing specific immunoproteins separately, produced by the methods comprising the steps of;
immunizing chicks by administering with 1 ml (10⁸/ml) antigen proteins of E. coli, Helicobacter pylori, Salmonella enteritidis and Salmonella typhimurium, emulsified with emulsifying adjuvant in a 1:1 ratio, separately, which comprise the 0.5 ml antigen of nonliving E. coli 2.0×10⁸/ml and 0.5 ml of aluminium sulfate, 0.5 ml antigen of nonliving Salmonella enteritidis 2.0×10⁸/ml and 0.5 ml of aluminium sulfate, 0.5 ml antigen of nonliving Salmonella typhimurium 2.0×10⁸/ml and 0.5 ml of aluminium sulfate, and 0.5 ml antigen of nonliving Helicobacter pylori 2.0×10⁸/ml and 0.5 ml of aluminium sulfate in the 1:1 ratio accordingly,
immunizing the chicks with 1 ml each of the antigen proteins emulsified with the emulsifying adjuvant ISA25 in the 1:1 ratio, accordingly, for two times by 2 weeks interval,
and immunizing the grown egg laying hens with 0.5 ml each of emulsified antigen proteins, which comprise the 0.5 ml antigen of nonliving E. coli 2.0×10⁸/ml and the 0.5 ml of emulsifying adjuvant ISA25, the antigen proteins 0.5 ml antigen of nonliving Salmonella enteritidis 2.0×108/ml and the 0.5 ml of emulsifying adjuvant ISA25, 0.5 ml antigen of nonliving Salmonella typhimurium 2.0×10⁸/ml and the 0.5 ml of emulsifying adjuvant ISA25, and 0.5 ml antigen of nonliving Helicobacter pylori 2.0×10⁸/ml and the 0.5 ml of emulsifying adjuvant ISA25 accordingly, for two times by 3 month interval as a total of 5 times.

14. The foodstuff processed with milk, containing the anti-mixed bacteria specific immunoprotein described in one of the claim 11-13.

15. The food additives containing the anti-mixed bacteria specific immunoprotein described in one of thee claim 11-13.

16. The method for isolating the water-soluble protein containing IgY, comprising the steps of:
diluting the egg yolk separated from the egg containing mixed anti-pathogenic bacteria specific antibodies (IgY) with distilled water in a certain ratio, for the first time adding ammonium sulfate to the diluents of egg yolk and for the second time distilled water to separate the water-soluble specific immunoprotein and phospholipid, sitting for certain time, diluting the supernatant collected after removing the upper lipid layer again, sitting for certain times, and isolating/purifying specific immunoprotein,

17. The method for isolating the water-soluble protein containing IgY according to claim 16, wherein the said amount of ammonium sulfate is 3%-10%.

18. The method for isolating the water-soluble protein containing IgY according to claim 16, wherein the said first dilution ratio is 1:1

19. The method for isolating the water-soluble protein containing IgY according to claim 16, wherein the said second dilution ratio is 1:12, 1:18, 1:30, 1:42, 1:48, 1:60.
